(19) United States
(12) Patent Application Publication
(10) Pub. No.: US 2008/0032954 A1
(43) Pub. Date: Feb. 7, 2008

(54) Vascular Damaging Agents for Administration as an Intravenous Infusion

(76) Inventor: Anderson Joseph Ryan, Cheshire (GB)

Correspondence Address: LADAS & PARRY, 26 WEST 61ST STREET, NEW YORK, NY, 10023, US

(21) Appl. No.: 11/596,521

(22) PCT Filed: May 11, 2005
(86) PCT No.: PCT/GB05/01802

§ 371 Date: Jul. 24, 2007

(30) Foreign Application Priority Data
May 14, 2004 (GB) 0410817.1

(51) Int. Cl. A61K 31/661; A61P 35/00; A61P 9/00

(52) U.S. Cl. 514/114

(57) ABSTRACT

The invention concerns the use of a vascular damaging agent or a pharmaceutically acceptable salt thereof in the manufacture of a medicament for administration as an intravenous infusion to a warm-blooded animal such as a human over a time period of more than 1 hour for use in the production of a vascular damaging effect in said warm-blooded animal. The invention also concerns kits adapted for intravenous infusion of a vascular damaging agent over a time period of more than 1 hour and to methods for providing a vascular damaging effect in a warm-blooded comprising administering a vascular damaging agent to the warm-blooded animal over a time period of more than 1 hour.

The present invention relates to a method for the production of a vascular damaging effect in a warm-blooded animal such as a human, more particularly to the use of a vascular damaging agent for the treatment of a cancer involving a solid tumour, in which a vascular damaging agent in administered to a warm blooded animal such as a human as an intravenous infusion over a time period of more than 1 hour.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Formation of new vasculature by angiogenesis is a key pathological feature of several diseases (J. Folkman, New England Journal of Medicine 333, 1757-1763 (1995)). For example, for a solid tumour to grow it must develop its own blood supply upon which it depends critically for the provision of oxygen and nutrients; if this blood supply is mechanically shut off the tumour undergoes necrotic death. Neovascularisation is also a clinical feature of skin lesions in psoriasis, of the invasive pannus in the joints of rheumatoid arthritis patients and of atherosclerotic plaques. Retinal neovascularisation is pathological in macular degeneration and in diabetic retinopathy.

Reversal of pathological neovascularisation by damaging the newly-formed vascular endothelium is expected to have a beneficial therapeutic effect. A number of vascular damaging agents (also known as vascular targeting agents) have been identified, for example combretastatin analogues such as combretastatin A4 phosphate and the Ajinomoto compound AC-7700 (also known as AVE8062A, and described in Nihei Y. et al. Japanese Journal of Cancer Research, 1999, 90, 1016-1025).

It has been found that the compounds described in International Patent Application numbers PCT/GB98/01977 (Publication No. WO 99/02166); PCT/GB99/04436 (Publication No. WO 00/40529); and PCT/GB/00099 (Publication No. WO 00/41669); have a selective damaging effect on newly formed vasculature as compared to the normal, established vascular endothelium of the host species. This is a property of value in the treatment of disease states associated with pathological angiogenesis such as cancer, diabetes, psoriasis, rheumatoid arthritis, Kaposi's sarcoma, haemangioma, acute and chronic nephropathies, atheroma, arterial restenosis, autoimmune diseases, acute inflammation, excessive scar formation and adhesions, endometriosis, dysfunctional uterine bleeding and ocular diseases with retinal vessel proliferation.

One compound described in PCT/GB98/01977 (Publication No. WO 99/02166) is N-acetylcolchinol-O-phosphate, (also know as (5S)-5-(acetylamino)-9,10,11-trimethoxy-6,7-dihydro-5H-dibenzo[a,c]cyclohepten-3-yl dihydrogen phosphate; Example 1 of WO 99/02166), which is referred to herein as ZD6126:

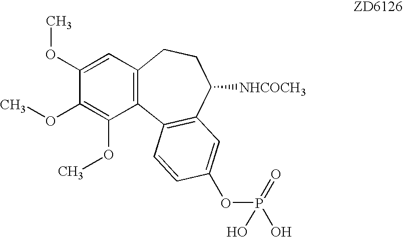

or a pharmaceutically acceptable salt thereof.

It is believed, though this is not limiting on the present invention, that vascular damaging agents such as ZD6126 damage newly formed vasculature, for example tumour vasculature, thus effectively reversing the process of angiogenesis by exploiting the differences between blood vessels in healthy tissue and the neovasculature of a tumour which is characterised by the presence of a rapidly proliferating endothelial cells and a chaotic network of highly permeable vessels. This is in contrast to anti-angiogenic agents which tend to be less effective once the vasculature resulting from angiogenesis has been established.

It is thought that vascular damaging agents, such as ZD6162 and the compounds described above, act to inhibit tubulin polymerisation, by de-stabilising microtubles, and thereby damaging neovasculature (formed as the result of inappropriate angiogenesis), because such neovasculature relies upon a tubulin cytoskeleton to maintain its 3-dimensional structure. This is in contrast to normal vasculature that has a well defined actin cytoskeleton. Accordingly, vascular damaging agents that inhibit tubulin polymerisation act to selectively damage the tubulin cytoskeleton of neovasculature whilst leaving intact normal vasculature (Blakey et al. Int. J. Radiation Oncology Biol. Phys. 54, (5), 1497-1502, 2002). Damage to pathological neovasculature, for example in a tumour, results in blood vessel congestion, loss of blood flow to the tumour and consequent tumour cell death due to nutrient deprivation and accumulation of toxic waste products. Blakey et al (Clinical Cancer Research, 8, 1974-1983, 2002) showed that ZD6126 disrupts the tubulin cytoskeleton causing rounding up and/or detachment of proliferating endothelial cells, which in tumours would lead to vessel occlusion with the consequence of rapid necrosis in the central regions of a solid tumour.

There is a wealth of pre-clinical data which shows that vascular damaging agents exhibit potent anti-tumour effects. However, more recently, pre-clinical and clinical data has emerged which suggests that vascular targeting agents can exhibit undesirable toxicity. In particular, there are reports of undesirable hemodynamic changes and cardiovascular toxicity, associated with the clinical use of vascular damaging agents, including blood pressure changes especially blood pressure increases, heart rate changes, ECG changes (such as T wave changes, ST segment changes, QT interval prolongation), increases in cardiac enzymes such as troponin T, troponin I or CK-MB), and more serious symptomatic ischemic cardiac events. For example, Cooney et al (Clinical Cancer research (10), 96-100) discusses the cardiovascular toxicity of combretastatin A4 phosphate in a phase I study of patients with advanced cancer. In this trial numerous hemodynamic and cardiac toxic events were observed following administration of combretastatin A4 phosphate, including potentially serious ischemic cardiac events such as a suspected myocardial infarction. Similarly, Tolcher et al (Abstract number 834, ASCO, 2003) found instances of asymptomatic hypotension following a 30 minute intravenous infusion of AVE8062A once weekly to patients. Furthermore, following a 10 minute intravenous infusion of ZD6126 once every three weeks, Gadgeel et al (Abstract number 438, ASCO, 2001) found that two patients exhibited grade 3 transient asymptomatic elevation in troponin I. One of these patients also had reversible grade 2 ischemic changes on ECG. The other patient had an increase in blood pressure one hour after dosing.

The undesirable hemodynamic changes and cardiovascular toxicity associated with vascular damaging agents currently means that patients with a compromised or dysfunctional cardiovascular system are excluded from receiving vascular damaging agents. In addition, even those patients without pre-existing cardiovascular risk factors may develop undesirable hemodynamic changes following treatment with vascular damaging agents. There is therefore a need to find a means for improving the therapeutic ratio between the clinically effective dose of vascular damaging agents and the dose which produces undesirable toxicity associated with hemodynamic changes and cardiac toxicity. Such an improvement in therapeutic index would enable patients to benefit from the potent anti-tumour effects of vascular targeting agents whilst avoiding or reducing the risk of occurrence of some of the clinically serious toxic side effects associated with vascular damaging agents. Such a means may therefore also be used to expand the patient population suitable for treatment with a vascular damaging agent.

Dowlati et al. (Cancer Research 62, 3408-3416, Jun. 2002) compare the dosing of combretastatin A4 to patients using a 10-minute intravenous infusion with a 60-minute intravenous infusion at three-week intervals. This study found no difference in the toxicity profile of either the 10- or the 60-minute infusion schedules.

Blakey et al. (Int. J. Radiation Oncology Biol. Phys. 54, (5), 1497-1502, 2002) found that dosing nude rats with 20 mg/kg of ZD6126 as a 24-hour subcutaneous infusion resulted in significant increases in toxicity but no increase in tumour necrosis compared to a single 5 to 10 second intravenous bolus dose of ZD6126. Furthermore, at a higher dose of 50 mg/kg a 24-hour subcutaneous infusion was not tolerated, in contrast to bolus dosing of the same dose of ZD6126.

PCT patent application publication No. WO 01/74369 discloses that administering the daily dose of a vascular damaging agent such as ZD6126 in divided doses enhances the vascular damaging effect produced by the vascular damaging agent. WO 01/74369 does not disclose anything about the hemodynamic effects of vascular damaging agents or how such changes may be controlled or mitigated.

PCT patent application publication No. WO01/74360 discloses the use of a combination of a vascular damaging agent and an anti-hypertensive as a means for controlling the blood pressure increases associated with the administration of the vascular damaging agent to a patient.

We have now surprisingly found that a prolonged intravenous infusion of a vascular damaging agent provides a vascular damaging effect and is expected to significantly reduce the incidence of undesirable hemodynamic changes and cardiac toxicity, and thereby increase the therapeutic ratio between the minimum therapeutically effective dose of the vascular damaging agent and the dose which produces such undesirable toxic events compared to the bolus administration of the same dose of the vascular damaging agent. Therefore, the invention is expected to reduce the risk of such hemodynamic changes and/or cardiac toxicity occurring during treatment. The invention may also allow patients currently excluded from receiving vascular damaging agents (for example patients with a compromised or dysfunctional cardiovascular system), to be treated with a vascular damaging agent thereby increasing the patient population suitable for such therapy.

According to a first aspect of the present invention there is provided a method for the production of a vascular damaging effect in a warm-blooded animal such as a human, which comprises administering to said animal as in intravenous infusion over a time period of more than 1 hour an effective amount of a vascular damaging agent or a pharmaceutically acceptable salt thereof.

According to a further aspect of the present invention there is provided the use of a vascular damaging agent or a pharmaceutically acceptable salt thereof in the manufacture of a medicament for administration as an intravenous infusion to a warm-blooded animal such as a human over a time period of more than 1 hour for use in the production of a vascular damaging effect in said warm-blooded animal.

According to a further aspect of the invention there is provided a vascular damaging agent or a pharmaceutically acceptable salt thereof for use in the production of a vascular damaging effect in a warm-blooded animal such as a human, characterised in that the vascular damaging agent is administered to said warm-blooded animal as an intravenous infusion over a time period of more than 1 hour.

The term “vascular damaging effect” is well known to those skilled in the art of vascular damaging agents and refers to damage to neovasculature that has formed as a result of inappropriate angiogenesis (i.e. pathological angiogenesis). The damage to such neovasculature may result in loss of structure of the neovasculature, reduced blood flow through the neovasculature, or leakage of blood from the neovasculature. For example, in a tumour the vascular damaging effect of the vascular damaging agent on the neovasculature supplying blood to the tumour results in, amongst other things, reduced blood flow to the tumour and rapid tumour necrosis. Accordingly the vascular damaging effect on, for example a tumour, includes reduction in blood flow to the tumour and/or the degree of tumour necrosis and/or a reduction in tumour size. The extent of vascular damage produced by a vascular damaging agent can be measured using known techniques, using in-vivo models such as those described in the Examples or other xenograft models such as those described in Davis et al, Cancer Research 62, 2002, 7247-7523 and WO99/02166; or using in-vivo imaging methods known in the art, for example CT scanning or MRI (Dowlati et al., Cancer Research 62, 3408-3416, June 2002), or PET (Anderson et al., Journal of Clinical Oncology 21, 2823-2830, August 2003).

Inappropriate or pathological angiogenesis is associated with a number of disease conditions, accordingly the vascular damaging effect produced by the present invention is expected to be useful in the prophylaxis and treatment of a wide range of disease states where inappropriate angiogenesis (pathological angiogenesis) occurs including cancer, (including leukaemia, multiple myeloma, lymphoma and particularly a cancer involving a solid tumour), diabetes, psoriasis, rheumatoid arthritis, Kaposi's sarcoma, haemangioma, acute and chronic nephropathies, atheroma, arterial restenosis, autoimmune diseases, acute inflammation, endometriosis, dysfunctional uterine bleeding and ocular diseases with retinal vessel proliferation including age-related macular degeneration.

As hereinbefore described, the reduction in hemodynamic changes associated with the intravenous administration of the vascular damaging agent is expected to reduce the incidence of cardiac toxicity associated with the vascular damaging agent. Accordingly, the invention is expected to be useful in the treatment of new patient populations which would hitherto have been excluded from treatment with a vascular damaging agent.

According to a further aspect of the invention there is provided a method for the production of a vascular damaging effect in a warm-blooded animal such as a human, said warm-blooded animal having a dysfunctional cardiac system, which method comprises administering to said animal as an intravenous infusion over a time period of more than 1 hour an effective amount of a vascular damaging agent or a pharmaceutically acceptable salt thereof.

According to a further aspect of the invention there in provided a method for the production of a vascular damaging effect in a warm-blooded animal such as a human, the method comprising selecting a warm-blooded animal such as a human with a dysfunctional cardiac system, and administering to said warm-blooded animal as an intravenous infusion over a time period of more than 1 hour an effective amount of a vascular damaging agent or a pharmaceutically acceptable salt thereof.

According to a further aspect of the present invention there is provided the use of a vascular damaging agent or a pharmaceutically acceptable salt thereof in the manufacture of a medicament for administration as an intravenous infusion to a warm-blooded animal such as a human over a time period of more than 1 hour for use in the production of a vascular damaging effect in said warm-blooded animal, and wherein the warm-blooded animal has a dysfunctional cardiac system.

According to a further aspect of the invention there is provided a vascular damaging agent or a pharmaceutically acceptable salt thereof for use in the production of a vascular damaging effect in a warm-blooded animal such as a human characterised in that the warm-blooded animal has a dysfunctional cardiac system and the vascular damaging agent is administered to said warm-blooded animal as an intravenous infusion over a time period of more than 1 hour.

References herein to a “dysfunctional cardiac system” in patients to be treated with a vascular damaging agent according to the invention refer to those patients with a pre-existing compromised cardiovascular system that may be further damaged or compromised by the rapid hemodynamic events associated with the acute (for example bolus) administration of a vascular damaging agent. Examples of patients with a dysfunctional cardiac system include those patients with an underlying cardiac disease, or an indicator of such a disease, for example a patient with arteriosclerosis, hypertension, cardiac arrhythmia, ECG abnormalities, elevation of cardiac enzymes, or a patient with a history of cardiac events, particularly ischemic events, for example angina or myocardial infarction, or a patient with one or more known risk factors for coronary heart disease such as smoking, diabetes mellitus, hyperlipidaemia, obesity, peripheral vascular disease, age over 65, abdominal aortic aneurysm or cerebrovascular accident.

In one embodiment of the invention the vascular damaging effect produced by the vascular damaging agent used in the method/use according to the invention provides an anti-tumour effect in a warm-blooded animal such as a human.

In a further embodiment of the invention the vascular damaging effect produced by method/use according to the invention provides an anti-cancer effect in a warm-blooded animal such as a human.

According to a further aspect of the invention there is provided a method for damaging pathological neovasculature associated with a disease state, for example a disease selected from cancer, (including leukaemia, multiple myeloma, lymphoma and particularly a cancer involving a solid tumour), diabetes, psoriasis, rheumatoid arthritis, Kaposi's sarcoma, haemangioma, acute and chronic nephropathies, atheroma, arterial restenosis, autoimmune diseases, acute inflammation, endometriosis, dysfunctional uterine bleeding and ocular diseases with retinal vessel proliferation including age-related macular degeneration in a warm-blooded animal such as a human, which comprises administering to said animal as an intravenous infusion over a time period of more than 1 hour an effective amount of a vascular damaging agent or a pharmaceutically acceptable salt thereof.

According to a further aspect of the invention there is provided the use of a vascular damaging agent or a pharmaceutically acceptable salt thereof in the manufacture of a medicament for administration as an intravenous infusion to a warm-blooded animal such as a human over a time period of more than 1 hour for use in the damage of pathological neovasculature associated with a disease state, for example a disease selected from cancer, (including leukaemia, multiple myeloma, lymphoma and particularly a cancer involving a solid tumour), diabetes, psoriasis, rheumatoid arthritis, Kaposi's sarcoma, haemangioma, acute and chronic nephropathies, atheroma, arterial restenosis, autoimmune diseases, acute inflammation, endometriosis, dysfunctional uterine bleeding and ocular diseases with retinal vessel proliferation including age-related macular degeneration in said warm-blooded animal.

According to a further aspect of the invention there is provided a vascular damaging agent or a pharmaceutically acceptable salt thereof for use in the damage of pathological neovasculature associated with a disease state, characterised in that the vascular damaging agent is administered to a warm-blooded animal such as a human as an intravenous infusion over a time period of more than 1 hour. Examples of diseases associated with pathological neovasculature are as hereinbefore defined.

The vascular damaging agents are particularly suitable for use in the provision of an anti-tumour effect in a warm blooded animal such as a human. Accordingly, a further aspect of the present invention provides a method for producing an anti-tumour effect in a warm-blooded animal such as a human, which comprises administering to said animal as in intravenous infusion over a time period of more than 1 hour an effective amount of a vascular damaging agent or a pharmaceutically acceptable salt thereof.

According to a further aspect of the invention there is provided the use of a vascular damaging agent or a pharmaceutically acceptable salt thereof in the manufacture of a medicament for administration as an intravenous infusion to a warm-blooded animal such as a human over a time period of more than 1 hour for use in the production of an anti-tumour effect in said warm-blooded animal.

The anti-tumour effect resulting from a method of treatment or use according to the present invention includes but is not limited to, inhibition of tumour growth, tumour growth delay, regression of tumour, shrinkage of tumour, increased time to regrowth of tumour on cessation of treatment, degree of tumour necrosis following administration of the vascular damaging agent, or slowing of disease progression. It is expected that when a method of treatment of the present invention is administered to a warm-blooded animal such as a human, in need of treatment for cancer involving a solid tumour, said method of treatment will produce an effect, as measured by, for example, one or more of: the extent of the anti-tumour effect, the response rate, the time to disease progression and the survival rate.

A particular embodiment of the invention provides a method for the treatment of a cancer (particularly a cancer involving a solid tumour) in a warm-blooded animal such as a human, which comprises administering to said animal as in intravenous infusion over a time period of more than 1 hour an effective amount of a vascular damaging agent or a pharmaceutically acceptable salt thereof.

Another embodiment of the invention provides the use of a vascular damaging agent or a pharmaceutically acceptable salt thereof in the manufacture of a medicament for administration as an intravenous infusion to a warm-blooded animal such as a human over a time period of more than 1 hour for use in the treatment of a cancer (particularly a cancer involving a solid tumour) in said warm-blooded animal.

The method and use according to the invention may be used to treat a wide range of cancers, and are expected to be particularly useful in the treatment of primary and recurrent solid tumours of, for example the colon, breast, prostate, lungs, skin, liver, head and neck, ovary, thyroid, gastrointestinal tract, colon or cervix.

Vascular damaging agents (VDAS) are agents which damage vasculature especially newly formed vasculature such as tumour vasculature and are well known to those skilled in the art. Suitable vascular damaging agents include tubulin binding agents, particularly tubulin binding agents that exhibit a vascular damaging effect, more particularly the vascular damaging agent is a tubulin binding, microtubule destabilising agent which exhibits a vascular damaging effect. Still more particularly the vascular damaging agent is a tubulin binding, microtubule destabilising agent which exhibits a vascular damaging effect and which induces acute hemodynamic changes (for example changes in blood pressure, heart rate or levels of cardiac enzymes) following a bolus administration to a warm-blooded mammal such as a human. Examples of vascular damaging agents include, but are not limited to combretastatin derivatives; colchinol derivatives; or benzimidazole derivatives that exhibit a vascular damaging effect. More particularly the vascular damaging agent is a combretastatin derivative or a colchinol derivative. Particular combretastatin derivatives include, for example combretastatin A-1, A-2, A-3, A-4, B-1, B-2, B-3, B-4, D-1, and D-2 as described in for example U.S. Pat. No. 4,490,726, U.S. Pat. No. 5,409,953 and U.S. Pat. No. 5,569,786 and pharmaceutically acceptable salts and prodrugs thereof, particularly phosphate prodrugs and pharmaceutically acceptable salts thereof, such as those described in WO 99/35150, WO 01/81355, WO 02/14329 and WO 02/22626; and synthetic combretastatin analogues such as those described in WO 00/35865, WO 00/48590, WO 01/12579, U.S. Pat. No. 5,430,062, U.S. Pat. No. 5,525,632, U.S. Pat. No. 5,674,906 and U.S. Pat. No. 5,731,353. A particular combretastatin is a phosphate prodrug of a combretastatin selected from combretastatin A4 phosphate, the Ajinomoto compound AC-7700 (also known as AVE8062A, Nihei Y. et al. Japanese Journal of Cancer Research, 1999, 90, 1016-1025) and combretastatin A1 diphosphate (also known as Oxi4503, Hua et al., Anticancer Res. 2003, 23(2B):1433-1440), or a pharmaceutically acceptable salt thereof. Examples of colchinol derivatives, include but are not limited to those described in International Patent Application No. PCT/GB98/01977 (Publication No. WO 99/02166) the entire disclosure of which document is incorporated herein by reference, and those described in International Patent Application No. PCT/GB99/04436 (Publication No. WO 00/40529) the entire disclosure of which document is incorporated herein by reference or the compounds described in WO02/04434, the compounds described in WO02/8213. Examples of other tubulin binding agents which have a vascular damaging effect when administered to a warm-blooded animal such as a human include the 2,3-disubstituted Benzo[b]thiophenes described in U.S. Pat. No. 5,886,025, U.S. Pat. No. 6,162,930, and U.S. Pat. No. 6,350,777; the 2,3-disubstituted benzo[b]furans described in WO 98/39323, the 2-3-disubstituted indoles described in WO 01/19794; the disubstituted dihydronaphthalenes described in WO01/68654; and benzimidazole derivatives with vascular damaging properties for example MN029 and the other benzimidazole derivatives described in WO 00/41669; and the Chalcone analogs described in WO 02/47604.

A particular VDA is ZD6126 or a pharmaceutically acceptable salt thereof.

Another particular VDA is combretastatin A4 phosphate, or a pharmaceutically acceptable salt thereof.

Another particular VDA is the compound AVE8062A, or a pharmaceutically acceptable salt thereof.

Another particular VDA is the compound Oxi4503, or a pharmaceutically acceptable salt thereof.

Another particular VDA is the compound MN029, or a pharmaceutically acceptable salt thereof.

In an embodiment of the invention the VDA is selected from a colchinol derivative or a combretastatin derivative, or a pharmaceutically acceptable salt thereof. For example the VDA is selected from ZD6126, combretastatin A4 phosphate, Oxi4503 and AVE8062A, or a pharmaceutically acceptable salt thereof.

In a particular embodiment the VDA is ZD6126 or a pharmaceutical salt thereof.

When the VDA is a pharmaceutically acceptable salt of ZD6126, suitable salts for use in the invention include salts formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

Suitably the infusion is administered as a continuous intravenous infusion over a time period of more than 1 hour. Accordingly the VDA is delivered to the warm-blooded animal continuously throughout the intravenous infusion period. The rate of administration of the VDA may be varied during the infusion period. However, generally the rate of administration of the vascular damaging agent will be substantially constant throughout the infusion period.

The intravenous infusion is administered over a time period of more than 1 hour, for example the infusion is suitably administered over a time period of about 1.5, 2, 2.5, 3, 3.5, 4, 4.5, 5, 6, 6.5, 7, 7.5, 8, 9, 10, 11 or 12 hours.

In an embodiment of the invention the intravenous infusion is administered over a time period of from 1.5 to 12 hours, more particularly from about 2 to about 10 hours, still more particularly from about 4 to about 8 hours, for example over a time period of about 6 hours.

For the avoidance of doubt the term ‘about’ in the description of time periods means the time given plus or minus 15 minutes, thus for example about 1.5 hours means 75 to 105 minutes. Elsewhere the term ‘about’ has its usual dictionary meaning.

A particular embodiment of the invention provides a method for the production of a vascular damaging effect in a warm-blooded animal such as a human, which comprises administering to said animal as an intravenous infusion over a time period of from about 4 to about 8 hours (for example about 6 hours) an effective amount of ZD6126 or a pharmaceutically acceptable salt thereof. In this embodiment the method suitably provides an anti-tumour effect, more particularly that the method provides a treatment of a cancer involving a solid tumour, as described hereinbefore.

Another particular embodiment of the invention provides the use of ZD6126 or a pharmaceutically acceptable salt thereof in the manufacture of a medicament for administration as an intravenous infusion to a warm-blooded animal such as a human over a time period of from about 4 to about 8 hours (for example about 6 hours) for use in the production of a vascular damaging effect in said warm-blooded animal. In this embodiment the medicament is particularly used to provide an anti-tumour effect, more particularly the medicament is used to provide a treatment of a cancer involving a solid tumour, as described hereinbefore.

The VDA is suitably used in the form of a pharmaceutical composition suitable for administration by intravenous infusion. Conveniently the VDA is used as a pharmaceutical composition comprising the VDA in association with a suitable a pharmaceutically acceptable liquid diluent or carrier. Pharmaceutical compositions suitable for intravenous administration are well known to those of ordinary skill in the art. For example, the VDA may be formulated as a sterile solution, suspension or emulsion in suitable pharmaceutically acceptable liquid medium. Suitable liquid media may be oil based or, particularly, an aqueous medium. The intravenous composition containing the VDA may optionally contain additional components conventionally used in such compositions, for example suspending agents, surfactants, viscosity modifiers, buffers and agents to adjust the pH of the composition.

The dose of VDA which is required for the therapeutic or prophylactic treatment of a particular disease state will necessarily be varied depending on the host treated, the route of administration and the severity of the illness being treated and the particular VDA that is used. Accordingly the optimum dosage may be determined by the practitioner who is treating any particular patient. For example when the VDA is ZD6126 it will normally be administered to a warm-blooded animal at a unit dose within the range 10-500 mg per square metre body area of the animal, for example approximately 0.3-15 mg/kg in a human. A unit dose in the range, for example, 0.3-15 mg/kg, such as 0.5-5 mg/kg is envisaged and this is normally a therapeutically-effective dose.

Use of an intravenous infusion of the VDA over a time period of more than 1 hour according to the present invention is expected to reduce the incidence or magnitude of undesirable hemodynamic events and/or cardiac toxicity compared to administration of the VDA as a short (less than about 10 minutes) infusion or bolus dose, whilst retaining a beneficial vascular damaging effect. Hemodynamic events that may be reduced using the method of the invention include changes in blood pressure or heart rate. Cardiac toxicity that may be reduced using the methods according to the invention include ECG changes such as QT interval prolongation and T wave changes; changes in cardiac enzymes such as troponin T, troponin I or CK-MB; acute coronary syndrome; or ischemic events such as myocardial infarction. The reduction in hemodynamic changes or cardiac toxicity may be assessed using known techniques, for example by monitoring blood pressure during and following administration of the vascular damaging agent, or by measuring the levels of cardiac enzymes such as troponins, or by monitoring for changes in ECG profiles or by monitoring for cardiac histopathological changes.

Accordingly, the method of the invention increases the therapeutic window between the minimum therapeutically effective dose of the VDA and the onset of clinically significant toxicity such as hemodynamic changes and cardiac toxicity. It is therefore expected that the method of the invention will enable patients to benefit from the anti-tumour effects of the VDA with a significantly reduced risk of experiencing a hemodynamic change or cardiac toxicity compared to dosing using known methods for the administration of VDAs. Accordingly, the invention is expected to provide a more clinically manageable toxicity profile compared with known dosage regimes, particularly bolus dosing of VDA's. As will be understood by those of ordinary skill in the art, the term “bolus dosing” or “bolus dose” used herein refers to the rapid intravenous administration of the vascular damaging agent as a single dose. Typically bolus dosing is performed by intravenous injection over a time period of less than 1 minute.

According to a further aspect of the invention there is provided a method for producing a vascular damaging effect in a warm-blooded animal such as a human that is in need of a treatment with a vascular damaging agent comprising administering a vascular damaging agent, or a pharmaceutically acceptable salt thereof, to said animal as an intravenous infusion over a time period of more than 1 hour in an amount sufficient to give a vascular damaging effect in said warm-blooded animal, whereby the incidence and/or magnitude of hemodynamic change is reduced compared to the administration of the same dose of vascular damaging agent as a bolus dose.

According to a further aspect of the invention there is provided a method for producing a vascular damaging effect in a warm-blooded animal such as a human that is in need of a treatment with a vascular damaging agent comprising administering a vascular damaging agent, or a pharmaceutically acceptable salt thereof, to said animal as an intravenous infusion over a time period of more than 1 hour in an amount sufficient to give a vascular damaging effect in said warm-blooded animal, whereby the incidence and/or magnitude of cardiac toxicity is reduced compared to the administration of the same dose of vascular damaging agent as a bolus dose.

According to a further aspect of the invention there is provided the use of a vascular damaging agent or a pharmaceutically acceptable salt thereof in the manufacture of a medicament for administration as an intravenous infusion to a warm-blooded animal such as a human over a time period of more than 1 hour for use in the production of a vascular damaging effect in said warm-blooded animal, and wherein the incidence and/or magnitude of hemodynamic changes caused by the administration of the vascular damaging agent to the warm blooded animal is lower than that observed when the same dose of vascular damaging agent is administered as a bolus dose.

According to a further aspect of the invention there is provided the use of a vascular damaging agent or a pharmaceutically acceptable salt thereof in the manufacture of a medicament for administration as an intravenous infusion to a warm-blooded animal such as a human over a time period of more than 1 hour for use in the production of a vascular damaging effect in said warm-blooded animal, and wherein the incidence of cardiac toxicity caused by the administration of the vascular damaging agent to the warm blooded animal is lower than that observed when the same dose of vascular damaging agent is administered as a bolus dose.

According to a further aspect of the present invention there is provided a vascular damaging agent or a pharmaceutically acceptable salt thereof for use in the production of a vascular damaging effect in a warm-blooded animal such as a human, characterised in that the vascular damaging agent is administered to said warm-blooded animal as an intravenous infusion over a time period of more than 1 hour, and wherein the incidence and/or magnitude of hemodynamic changes caused by the administration of the vascular damaging agent to the warm blooded animal is lower than that observed when the same dose of vascular damaging agent is administered as a bolus dose.

According to a further aspect of the present invention there is provided a vascular damaging agent or a pharmaceutically acceptable salt thereof for use in the production of a vascular damaging effect in a warm-blooded animal such as a human, characterised in that the vascular damaging agent is administered to said warm-blooded animal as an intravenous infusion over a time period of more than 1 hour, and wherein the incidence of cardiac toxicity caused by the administration of the vascular damaging agent to the warm blooded animal is lower than that observed when the same dose of vascular damaging agent is administered as a bolus dose.

According to a further aspect of the present invention there is provided a kit comprising a VDA; and instructions for administration of the VDA as an intravenous infusion over a time period of more than 1 hour to a warm blooded animal such as a human.

According to a further aspect of the present invention there is provided a kit comprising a VDA; which kit is adapted for administration of the VDA as an intravenous infusion over a time period of more than 1 hour to a warm blooded animal such as a human.

Suitable VDA's and intravenous infusion times for use in the kits of the invention are as hereinbefore defined in relation to the method and use according to the invention. Suitable pharmaceutical compositions are as hereinbefore described.

In an embodiment of the kit according to the invention, the VDA is present in the kit in the form of a pharmaceutical composition suitable for intravenous administration. Pharmaceutical compositions comprising a VDA suitable for intravenous administration are as hereinbefore described in relation to the method and use according to the invention.

The use/method of treatment of the present invention as defined herein may be applied as a sole therapy or may involve, in addition to a vascular damaging agent administered in divided doses, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the VDA administered as an intravenous infusion over a time period of more than 1 hour, may be: surgery, radiotherapy or chemotherapy. Such chemotherapy may include the following categories of therapeutic agent:

(i) antiangiogenic agents (for example linomide, inhibitors of integrin αvβ3 function, angiostatin, endostatin, razoxin, thalidomide) and including vascular endothelial growth factor (VEGF) receptor tyrosine kinase inhibitors (RTKIs) (for example those described in International Patent Applications Publication Nos. WO 97/22596, WO 97/30035, WO 97/32856 and WO 98/13354 the entire disclosure of which documents is incorporated herein by reference, also for example those described in International Patent Application Publication No. WO 00/47212 the entire disclosure of which is incorporated herein by reference);

(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example epidermal growth factor (EGF), platelet derived growth factor and hepatocyte growth factor such inhibitors include growth factor antibodies, growth factor receptor antibodies, tyrosine kinase inhibitors and serine/threonine kinase inhibitors);

(iii) biological response modifiers (for example interferon);

(iv) antibodies (for example edrecolomab); and

(v) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); enzymes (for example asparaginase); thymidylate synthase inhibitors (for example raltitrexed); topoisomerase inhibitors (for example epipodophyllotoxins like etoposide and teniposide, amsacrine, topotecan, irinotecan).

Vascular damaging agents, such as ZD6126, or pharmaceutically acceptable salts thereof, are particularly suitable for use in combination with radiotherapy and one or more anti-tumour therapeutic agents selected from a platinum anti-tumour agent (for example cisplatin, carboplatin or oxaliplatin); a taxane (for example paclitaxel or docetaxel); a nitric oxide synthase inhibitor (for example a derivative of arginine, ornithine, lysine, citrulline, S-alkylthioureas or aminoguanidine); a vinca alkaloid (for example vincristine); an antimetabolite (for example gemcitabine); a fluoropryimidine (for example 5-FU and derivatives thereof such as capcitabine, tegafur or TS-1); a topoisomerase inhibitor (for example irinotecan); an epidermal growth factor receptor tyrosine kinase inhibitor (for example Iressa (gefitinib) or Tarceva (erlotinib). Examples of suitable combination treatments utilising a VDA such as ZD6126 and radiotherapy and/or one or more of the therapeutic agents described above are described in for example WO 00/48591, WO 01/74368, WO 03/088971 and WO 04/032937. The combination therapies described in these patent applications may be used in the present invention provided that the VDA is administered as an intravenous infusion over a time period of more than 1 hour as described herein. The additional anti-cancer therapies such as the chemotherapies described herein and/or radiotherapy may be administered substantially simultaneously, sequentially or separately with the intravenous administration of the vascular damaging agent.

The invention is illustrated by the following examples. In the examples the following abbreviations have been used:

H&E: Hematoxylin-and-eosin;

IV: Intravenous;

PBS: Phosphate buffered saline;

Inf. Infusion

### BRIEF DESCRIPTION OF FIGURE

FIG. 1 shows the tumour necrosis score in each of the treatment groups shown on the X-axis in FIG. 1. The treatment groups represent rats that were given a prolonged intravenous infusion of ZD6126 for the time period shown; or a control group (first column-no ZD6126); or a first comparative group (second column) that received a bolus dose of ZD6126; or a second comparative group (third column) which received an intravenous infusion of ZD6126 over 0.25 hours. In FIG. 1 the term “h” on the X-axis refers to hours.

### EXAMPLE 1

### Calu-6 Human Tumour Xenograft Model in Nude Rats

2.0×10⁷ Calu-6 cells were inoculated subcutaneously in 0.2 ml of RPMI-1640 medium into the right flank of female Nude rats. When tumours reached 500 to 1000 mm³ rats were randomised in 8 groups of 7 rats.

The treatment schedule of ZD6126 consisted of a continuous IV infusion of a total dose of 35.0 mg/kg delivered during different periods: 0.25, 1, 2, 6, 12 or 24 hours for groups 3, 4, 5, 6, 7 and 8, respectively as shown in Table 1. One group received a continuous IV infusion of PBS over a time period of 0.25 hour, and a further group was treated with a single IV bolus of ZD6126 at 35.0 mg/kg.

The treatment schedule is summarized in Table 1 below:

TABLE 1

| | | | | | | Period of |
|---|---|---|---|---|---|---|
| | No. | | Dose | Adm. | Mode | infusion |
| Groups | Rats | Treatment | (mg/kg) | route | injection | (hours) |
| 1 | 7 | Vehicle | — | — | Continuous | 0.25 |
| | | | | | infusion | |
| 2 | 7 | ZD6126 | 35.0 | IV | Bolus | — |
| | | | | | injection | |
| 3 | 7 | ZD6126 | 35.0 | IV | Continuous | 0.25 |
| | | | | | infusion | |
| 4 | 7 | ZD6126 | 35.0 | IV | Continuous | 1.00 |
| | | | | | infusion | |
| 5 | 7 | ZD6126 | 35.0 | IV | Continuous | 2.00 |
| | | | | | infusion | |
| 6 | 7 | ZD6126 | 35.0 | IV | Continuous | 6.00 |
| | | | | | infusion | |
| 7 | 7 | ZD6126 | 35.0 | IV | Continuous | 12.00 |
| | | | | | infusion | |
| 8 | 7 | ZD6126 | 35.0 | IV | Continuous | 24.00 |
| | | | | | infusion | |

Immediately after the end of the infusion treatment period blood was collected and plasma prepared to determine drug levels. Twenty four hours after treatment was started, 3 rats per group were sacrificed the tumor from each rat was be excised and fixed in 10% formaldehyde buffer. The extent of tumour necrosis was determined following H&E staining of tumour sections using the method described on page 1975 in Blakey et al (Clinical Cancer Research, 8, 1974-1983, 2002) to give the tumour necrosis scores shown in Table 1.
Results:

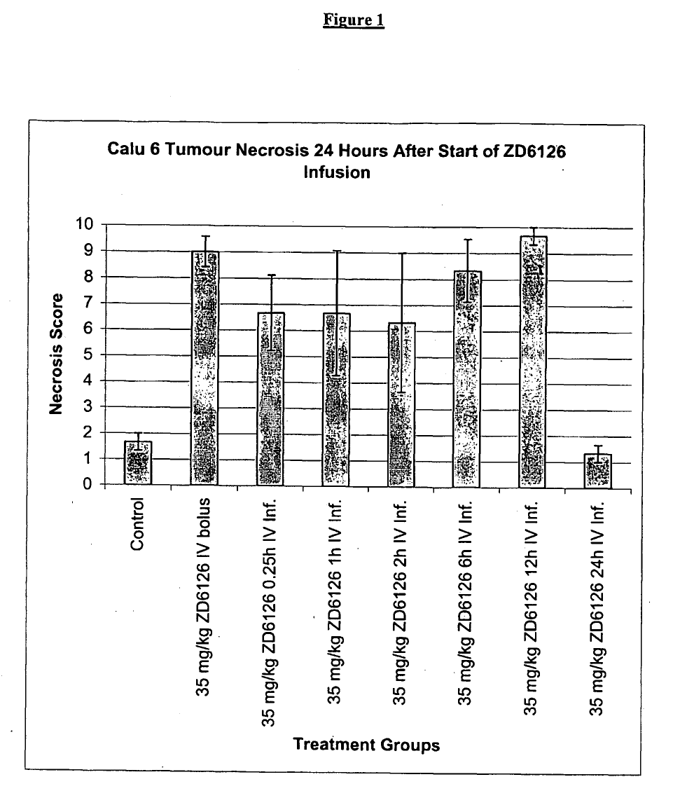

The results are summarised in FIG. 1. FIG. 1 shows the effect of ZD6126 on tumour necrosis for each treatment group. The results show that a continuous infusion of ZD6126 over a time period of up to 12 hours produces a similar antitumour effect, by way of the observed tumour necrosis, to that achieved using the same dose but administered as a single bolus intravenous dose.

1-14. (canceled)

15. A method for the production of a vascular damaging effect in neovasculature associated with a disease state in a human or a warm-blooded animal, which comprises administering to said human or warm-blooded animal as in intravenous infusion over a period of more than 1 hour an effective amount of a vascular damaging agent or a pharmaceutically acceptable salt thereof.

16. A method for the production of a vascular damaging effect in neovasculature associated with a disease state in a human or a warm-blooded animal, which comprises administering to said human or warm-blooded animal as in, intravenous infusion over a period of more than 1 hour an effective amount of a vascular damaging agent or a pharmaceutically acceptable salt thereof, and wherein the human or warm-blooded animal has a dysfunctional cardiac system or a cardiac risk factor.

17. The method according to claim 15, wherein the disease state includes a tumour.

18. The method according to claim 17, wherein the tumour is a solid tumour.

19. The method according to claim 15, wherein the disease state is cancer.

20. The method according to claim 15, wherein the intravenous infusion is over a period of from about 1.5 hours to about 24 hours.

21. The method according to claim 15, wherein the intravenous infusion is over a period of from about 4 hours to about 8 hours.

22. The method according to claim 15, wherein the intravenous infusion is over a period of about 6 hours.

23. The method according to claim 15, wherein the vascular damaging agent is a tubulin binding, ricrotubule destabilising agent.

24. The method according to claim 15, wherein the vascular damaging agent is selected from ZD6126, Oxi4503, AVE8062A, Combretastatin A4 phosphate and MN029, or a pharmaceutically acceptable salt thereof.

25. The method according to claim 15, wherein the vascular damaging agent is ZD6126 or a pharmaceutically acceptable salt thereof.

26. The method according to claim 15, wherein the human or warm-blooded animal is also treated substantially simultaneously, sequentially or separately with a therapy selected from an anti-cancer therapy and radiotherapy.

27. The method according to claim 15, wherein said disease state is selected from leukaemia, multiple myeloma, lymphoma, diabetes, psoriasis, rheumatoid arthritis, Kaposi's sarcoma, haemangioma, acute and chronic nephropathies, atheroma, arterial restenosis, autoimmune diseases, acute inflammation, endometriosis, dysfunctional uterine bleeding and ocular diseases with retinal vessel proliferation including age-related macular degeneration.

28. The method according to claim 16, wherein the disease state is cancer.

29. The method according to claim 16, wherein the intravenous infusion is over a period of from about 1.5 hours to about 24 hours.

30. The method according to claim 16, wherein the vascular damaging agent is a tubulin binding, microtubule destabilising agent.

31. The method according to claim 16, wherein the vascular damaging agent is selected from ZD6126, Oxi4503, AVE8062A, Combretastatin A4 phosphate and MN029, or a pharmaceutically acceptable salt thereof.

32. The method according to claim 16, wherein the vascular damaging agent is ZD6126 or a pharmaceutically acceptable salt thereof.

33. The method according to claim 16, wherein the human or warm-blooded animal is also treated substantially simultaneously, sequentially or separately with a therapy selected from an anti-cancer therapy and radiotherapy.

34. A kit comprising a vascular damaging agent, and instructions for administration of the formulation as an intravenous infusion over a period of more than 1 hour to a human or warm-blooded animal.
